(19) United States
(12) Patent Application Publication
(10) Pub. No.: US 2023/0201105 A1
(43) Pub. Date: Jun. 29, 2023

(54) Lip Gloss Formulation

(71) Applicant: Guangen Xiang, Foothill Ranch, CA (US)

(72) Inventor: Guangen Xiang, Foothill Ranch, CA (US)

(21) Appl. No.: 17/645,756

(22) Filed: Dec. 23, 2021

(51) Int. Cl. A61K 8/9789; A61K 8/891; A61K 8/34; A61K 8/86; A61K 8/81; A61K 8/31; A61K 8/44; A61K 8/67; A61K 8/36; A61K 8/37; A61Q 1/06

(52) U.S. Cl. CPC A61K 8/9789; A61K 8/891; A61K 8/345; A61K 8/86; A61K 8/8111; A61K 8/31; A61K 8/44; A61K 8/678; A61K 8/36; A61K 8/375; A61K 8/8152; A61K 8/347; A61Q 1/06; A61K 2800/524; A61K 2800/43; A61K 2800/591

(57) ABSTRACT

Disclosed in the present invention is a formulation of lip gloss, comprising at least main ingredients, stabilizers, preservatives, flavours and some additives. The ingredients comprise Centella asiatica extract, dimethicone, Sodium Hyaluronate, Glycerin, Polysorbate 60, Polyisobutene, Squalane, Arginine, Tocopherol. Stabilizers comprise Sorbitan Isostearate, 1, 2-Hexanediol, Hydroxyacetophenone, water, Acrylates Copolymer, Polyacrylate-13 and Polysorbate 20 or water and Acrylates Copolymer by weight. Preservatives comprise Hydroxyacetophenone and 1,2-Hexanediol. Additionally, the said invention may comprise at least CI 15850, CI 19140, CI 17200, CI 15985, CI 42090, CI 15510 and CI 16035 as additives in designated amount by weight. The said invention comes with a shiny finish, long lasting or transfer proof.

### TECHNICAL FIELD OF THE INVENTION

The present invention relates to makeup industry, and in particular to a lip gloss formulation that comes with a shiny finish, long lasting or transfer resistance as opposed to the traditional formulation on lip gloss that is shorter lasting, easy to transfer when lip touches food, cup or other objects.

### BACKGROUND ART

Traditionally, lip gloss is long lasting if it is applied but not accidentally transferred. In the present invention, the formulated lip gloss is longer lasting since it is not as easily transferred. Just like the name “lip gloss”, the biggest challenge for traditional formulation on lip gloss is that the current art is shorter lasting, easy to transfer when lip touches food, cup or other objects; then people will need to re-apply over and over again throughout the day.

Lipsticks and lip glosses are often intended to bring high gloss and shine when applied on lips. This is generally obtained by use of oils, esters, and silicones helping to form a homogeneous coating on lips and enhancing the light reflection by increasing the refractive index of the lipstick film. These ingredients also help the cohesion of a formulation, especially when using fillers and larger pigments that disrupt the cohesion of a lipstick during molding.

A makeup product is used to bring color to the skin. It is also recognized for its ease of application, its makeup result, its color, its dullness, its homogeneity, but also for the makeup over time, especially throughout the day. It is known to those skilled in the art to provide these bolding properties by formulating compositions comprising silicone resins, polyacrylates, or latices, which unfortunately have the tendency to make the texture of the product thicker, and thus make the application on the skin more difficult; the product stretches or spreads less easily on the face or body. It therefore appears necessary to provide a technical solution for formulating products, and more particularly emulsions and lip gloss, having a satisfactory makeup performance, while maintaining ease of application.

In addition, many cosmetic compositions, including pigmented cosmetics such as lipsticks or lip colors, have been formulated in an attempt to possess long-wearing properties upon application. Unfortunately, many of these compositions do not generally possess good long-wear/transfer-resistance properties as well as good appearance properties (for example, satin properties) and/or application properties. With respect to lip compositions, commercial products containing silicon resins such as MQ resins are known. Such products are known to provide good long wear properties/transfer-resistance. However, such products possess poor application properties and/or poor feel upon application (for example, feels rough). Typically, a second composition (topcoat) is separately applied to such products to improve poor properties of the compositions to make the products acceptable to consumers. However, topcoat compositions tend to decrease the long-wear/transfer-resistance properties of the lip compositions, thereby rendering the long-wear/transfer resistance composition less acceptable to consumers and less acceptable for their intended purpose. Thus, there remains a need for improved lip gloss formulations having improved cosmetic or makeup properties, particularly good transfer-resistance, feel and/or glossy characteristics upon application.

U.S. Ser. No. 10/786,439B2 filed in 2018 by L'Oreal SA entitled “Satin Lip Compositions.” It claims a satin lip composition comprising at least one silicone resin comprising at least one T unit, at least one silicone acrylate copolymer, at least one silicone wax, at least one volatile hydrocarbon oil, wherein the at least one volatile hydrocarbon oil is present in an amount less than or equal to 25% by weight based on the total weight of the composition, and at least one non-volatile hydrocarbon oil having a molecular weight greater than 400 g/mol which is a C3-05 polyalkene, wherein the composition has average gloss properties, measured at 60°, of 20 to 60 and wherein the silicone resin(s) comprising at least one T unit and silicone acrylate copolymer(s) are present in a weight ratio of from about 5:1 to about 1.5:1.

US20190099361A1 filed by Kay Mary Inc in 2012 entitled “Cosmetic compositions.” It claims a method for treating skin comprising topically applying a composition comprising: (a) an effective amount of a combination of Centella asiatica extract, tetradecyl aminobutyroylvalylamino butyric urea trifluoroacetate, and tripeptide-1 to promote the production of hyaluronic acid in skin and to promote the production of fibronectin and laminin in skin; (b) one or more emulsifiers; (c) water; (d) glycerin; and (e) sunflower (Helianthus annuus) seed oil, to skin in need of treatment, wherein topical application of the composition increases the production of hyaluronic acid in skin and/or increases the production of fibronectin and laminin in skin. Many competitors have made long lasting matte or liquid lipstick, but none have been able to develop transfer resistance lip gloss as the current invention has.

An International Patent Filing No. WO2009080963A2 filed by L'oreal in 2008 entitled “Cosmetic process using a composition comprising a siloxane resin and a nonionic silicone surfactant.” Cosmetic process for making up and/or caring for keratin materials, comprising the application to said keratin materials, in particular the skin, of a composition, in the form of an emulsion, comprising in a physiologically acceptable medium: i) at least one siloxane resin comprising the units: <EMI ID 59.1> (ii) (R <2>2SiO 2/2) b <EMI ID=59.2> (iv) (SiO4/2) d with R <1>, R <2> and R <3> independently representing an alkyl group having from 1 to 8 carbon atoms, an aryl group, a carbinol group or an amino group, a being between 0.05 and 0, 5, b being between zero and 0.3, c being greater than zero, d being between 0.05 and 0.6, a+b+c+d=1, provided that more than 40 mol % of R<3> groups of the siloxane resin are propyl groups, and ii) at least one silicone nonionic surfactant.

However, the above prior art documents only relates to the claimed lip gloss invention but do not disclose the same values or ratios as claimed by lip gloss invention. As such, we can infer that the new innovative formation of shiny lip gloss is novel over the existing prior art in that no single prior art patent has claimed all the components of the supplement.

From our market research, there is no brand or formula till now that can create a solution to keep the lip gloss shinny finish while making it transfer resistance and long lasting to a certain degree. The new innovative formulation solves this challenge. It is a formula that makes the lip gloss shinny while long lasting/transfer resistance.

### OBJECT OF THE INVENTION

The main object of the present invention is to develop a lip stick formulation that has a shiny lip gloss and which is long lasting or transfer resistance as opposed to the traditional formulation on lip gloss that is shorter lasting, easy to transfer when lip touches food, cup or other objects, and which eliminates the need for users to re-apply the lip over and over again throughout the day.

### SUMMARY OF INVENTION

The present invention discloses a new innovative formulation of lip gloss comprising dimethicone, Emollient, Polysorbate 60, Polyisobutene, Squalane, Arginine, Centella asiatica extract, Tocopherol and preservatives by weight. Also disclosed is a set preservatives and stabilizers comprising Sodium Hyaluronate, Sorbitan Isostearate, 1, 2-Hexanediol, Hydroxyacetophenone, water, Acrylates Copolymer, Polyacrylate-13, Polysorbate 20, and Flavor by weight.

Additionally, the said invention may comprise the following flavours: CI 15850, CI 19140, CI 17200, CI 15985, CI 42090, CI 15510 and CI 16035 in designated amount by weight. The said invention comes with a shiny finish, long lasting or transfer resistance.

Yet in the same disclosure the present invention further provides a formulation of lip gloss which has a plurality functional ingredients such as; solvent, emulsifier, emulsifying such as solvent, emulsifier, emulsifying stabilizer, skin feel regulators, antioxidants, emollients, moisturizer, aromatics and colorant.

The said invention comes with a shiny finish and long lasting or transfer resistance as opposed to the traditional formulation on lip gloss that is shorter lasting, easy to transfer when lip touches food, cup or other objects. The current invention eliminates the need for users to re-apply the lip over and over again throughout the day.

### DETAILED DESCRIPTION

As used herein, the expression “at least” means one or more and thus includes individual ingredients or components as well as mixtures/combinations of ingredients or components.

Other than in the operating examples, or where otherwise indicated, all numbers expressing quantities of ingredients and/or reaction conditions are to be understood as being modified in all instances by the term “between,” meaning within percentage range of the indicated number including the number itself.

The present invention teaches various combination of ingredients used in a wide range of applications to lip makeup. In one embodiment, the invention discloses a new innovative formulation of shiny lip gloss comprising at least, dimethicone, Sodium Hyaluronate, Glycerin, Polysorbate 60, Polyisobutene, Squalane, Arginine, Centella asiatica extract, Tocopherol as ingredients in addition to stabilizers, preservatives by weight and flavours. In another embodiment, the invention describes a set preservatives comprising at least Sorbitan Isostearate, 1, 2-Hexanediol, Hydroxyacetophenone, water, Acrylates Copolymer, Polyacrylate-13, Polysorbate 20, and Flavor by weight.

Preferably, a formulation of shiny lip gloss comprising the following ingredients: dimethicone of between 6-11% by weight, Glycerin of between 5-11% by weight, Polysorbate between 20 (1-2% by weight), Polysorbate 60 (1-2% by weight), Squalane of between 2-10% by weight, Arginine of between 0.1-0.8% by weight, 1,2-Hexanediol of between 0.1-0.8% by weight, Centella asiatica extract of between 0.1-0.8% by weight, Tocopherol of between 0.1-0.8% by weight, Sodium Hyaluronate of between 0.01-0.1% by weight and Flavor of between 0.01-0.1% by weight as shown in Example 1 below.

Preferably, a formulation of shiny lip gloss comprising water of between 50-70% by weight and at least one of the following other stabilizers: Polyacrylate-13 (1-2% by weight), Polyisobutene of 0.1 (0.8% by weight), Polysorbate 20 (1-0.8% by weight) and Sorbitan Isostearate of between 0.01-0.1% by weight.

Preferably, a formulation of shiny lip gloss comprising at least water of between 50-70% by weight and Acrylates Copolymer of between 0.5-2% by weight as preservative.

Preferably, a formulation of shiny lip gloss comprising at least water of between 50-70% by weight and Hydroxyacetophenone of between 0.1-0.8% by weight.

### Example Table 1: Product Ingredients List

| Serial | | |
|---|---|---|
| Number | Inci Name | Calc. As % |
| 1 | Water | 50-70 |
| 2 | Dimethicone | 6-11 |
| 3 | Emollient | 5-11 |
| 4 | Acrylates Copolymer | 0.5-2 |
| 5 | Polysorbate 20 | 1-2 |
| 6 | Polysorbate 60 | 1-2 |
| 7 | Polyacrylate-13 | 1-2 |
| | Polyisobutene | 0.1-0.8 |
| | Polysorbate 20 | 0.1-0.8 |
| | Sorbitan Isostearate | 0.01-0.1 |
| 8 | Squalane | 2-10 |
| 9 | Arginine | 0.1-0.8 |
| 11 | 1,2-Hexanediol | 0.1-0.8 |
| 12 | Hydroxyacetophenone | 0.1-0.8 |
| 13 | Centella Asiatica Extract | 0.1-0.8 |
| 14 | Tocopherol | 0.1-0.8 |
| 15 | Sodium Hyaluronate | 0.01-0.1 |
| 16 | Flavor | 0.01-0.1 |

In a similar but related embodiment, the invention discloses a formulation of shiny lip gloss that may further comprises at least one of the following flavours: CI 15850, CI 19140, CI 17200, CI 15985, CI 42090, CI 15510 and CI 16035 in the amount of 0.01-0.1% each by weight as additional ingredients as shown in Example 2 below.

### Example Table 2: Flavours

May Contain the Following Flavours

| Serial | | |
|---|---|---|
| Number | Inci Name | Calc. As % |
| 1 | CI 15850 | 0.01-0.1 |
| 2 | CI 19140 | 0.01-0.1 |
| 3 | CI 17200 | 0.01-0.1 |
| 4 | CI 15985 | 0.01-0.1 |
| 5 | CI 42090 | 0.01-0.1 |
| 6 | CI 15510 | 0.01-0.1 |
| 7 | CI 16035 | 0.01-0.1 |

The present invention further provides a formulation of lip gloss which has a plurality functional ingredients such as; solvent, emulsifier, emulsifying such as solvent, emulsifier, emulsifying stabilizer, skin feel regulators, antioxidants, emollients, moisturizer, aromatics and colorant.

Specifically, the ingredient with solvent function is about 65% by weight while the ingredient with emulsifier about 2% by weight. The ingredient with emulsifying stabilizer function is in the range of between 0.56-2.1% by weight. The ingredient with skin feel regulator function is in the range of between 0.1-5% by weight. Whereas the ingredient with antioxidants function is in the range of between 0.1-0.2% by weight, the ingredient with emollients function is in the range of between 8-11% by weight. In the same formulation of lip gloss whereas the ingredient with moisturizer function is in the range of between 0.05-0.2% by weight, that with aromatics function is about 0.03% by weight. The ingredient with colorant function is in the range of between 0.1-0.3% by weight as shown the table below:

### Example Table 3—Functional Ingredients

Calc. As %

| No. | Functional Ingredient | Lowest | Highest |
|---|---|---|---|
| 1 | Solvent | | 65 |
| 2 | Emollients | 8 | 11 |
| 3 | Emulsifying stabilizers | 0.56 | 2.1 |
| 4 | Emulsifier | | 2 |
| 5 | Skin Feel Regulator | 0.1 | 5 |
| 6 | Moisturizer | 0.05 | 0.2 |
| 7 | Antioxidants | 0.1 | 0.2 |
| 8 | Aromatics | | 0.03 |
| 9 | Colorant | 0.1 | 0.3 |
| | Total Formulation | | 100 |

The said invention comes with a shiny finish and long lasting or transfer resistance as opposed to the traditional formulation on lip gloss that is shorter lasting, easy to transfer when lip touches food, cup or other objects. The current invention eliminates the need for users to re-apply the lip over and over again throughout the day.

The aforementioned combination of ingredients can be used in a wide range of applications to lip makeup. For instance, formulations having any of the combinations can be topically applied to lip in need of such makeup.

### INDUSTRIAL APPLICATION

The present invention applies to makeup industry. It is particularly useful in beauty industry where a lip gloss that is long lasting or transfer resistance is required as opposed to the traditional formulation on lip gloss that is shorter lasting, easy to transfer when lip touches food, cup or other objects, and which eliminates the need for users to re-apply the lip over and over again throughout the day.

What is claimed is:

1. A formulation of lip gloss, comprising at least one of the following ingredients:
a. Centella asiatica extract in specified amount by weight,
b. Dimethicone in specified amount by weight,
c. Glycerin in specified amount by weight,
d. Polysorbate 60 in specified amount by weight,
e. Polyisobutene in specified amount by weight,
f Squalane in specified amount by weight,
g. Arginine in specified amount by weight,
h. Tocopherol in specified amount by weight,
i. Sodium Hyaluronate in specified amount by weight,
j. Stabilizers in specified amount by weight,
k. preservatives by weight,
l. flavours in specified amount by weight,
m. additives in specified amount by percentage, and
n. a plurality of functional ingredients within the formulation.

2. The formulation of lip gloss according to claim 1, wherein the amount of Centella asiatica extract in the formulation is in the range of between 1-2% by weight.

3. The formulation of lip gloss according to claim 1, wherein the amount of dimethicone in the formulation is in the range of between 6-11% by weight.

4. The formulation of lip gloss according to claim 1, wherein the amount of Glycerin in the formulation is between 5-11% by weight.

5. The formulation of lip gloss according to claim 1, wherein the amount of Polysorbate 60 in the formulation is in the range of between 1-2% by weight.

6. The formulation of lip gloss according to claim 1, wherein the amount of Polyisobutene 0.1 in the formulation is in the range of between 0.8% by weight.

7. The formulation of lip gloss according to claim 1, wherein the amount of Squalane in the formulation is in the range of between 2-10% by weight.

8. The formulation of lip gloss according to claim 1, wherein the amount of Arginine in the formulation is in the range of between 0.1-0.8% by weight.

9. The formulation of lip gloss according to claim 1, wherein the amount of Tocopherol in the formulation is in the range of between 0.1-0.8% by weight.

10. The formulation of lip gloss according to claim 1, wherein the amount of Sodium Hyaluronate in the formulation is in the range of between 0.01-0.1% by weight.

11. The formulation of lip gloss according to claim 1, wherein the stabilizers further comprise Sorbitan Isostearate, 1, 2-Hexanediol, Hydroxyacetophenone, water, Acrylates Copolymer, Polyacrylate-13 and Polysorbate 20.

12. The formulation of lip gloss according to claim 11, wherein the amount of Sorbitan Isostearate in the formulation is in the range of between 0.01-0.1% by weight.

13. The formulation of lip gloss according to claim 11, wherein the amount of 1,2-Hexanediol in the formulation is in the range of between 0.1-0.8% by weight.

14. The formulation of lip gloss according to claim 11, wherein the amount of water in the formulation is in the range of between 50-70% by weight.

15. The formulation of lip gloss according to claim 11, wherein the amount of Acrylates Copolymer in the formulation is in the range of between 0.5-2% by weight.

16. The formulation of lip gloss according to claim 11, wherein the amount of Polyacrylate-13 in the formulation is in the range of between 1-2% by weight.

17. The formulation of lip gloss according to claim 11, wherein the amount of Polysorbate 20 in the formulation is in the range of between 1-2% by weight.

18. The formulation of lip gloss according to claim 1, wherein the preservatives further comprise water and.

19. The formulation of lip gloss according to claim 18, wherein the amount of water in the formulation is in the range of between 50-70% by weight.

20. The formulation of lip gloss according to claim 1, wherein the stabilizers further comprise water and Acrylates Copolymer by weight.

21. The formulation of lip gloss according to claim 20, wherein the amount of water in the formulation is in the range of between 50-70% by weight.

22. The formulation of lip gloss according to claim 20, wherein the amount of Acrylates Copolymer in the formulation is in the range of between 0.5-2% by weight.

23. The formulation of lip gloss according to claim 1, wherein the preservatives further comprise 1,2-Hexanediol and Hydroxyacetophenone by weight.

24. The formulation of lip gloss according to claim 23, wherein the amount of 1, 2-Hexanediol in the formulation is in the range of between 0.1-0.8% by weight.

25. The formulation of lip gloss according to claim 23, wherein the amount of Hydroxyacetophenone in the formulation is in the range of between 0.1-0.8% by weight.

26. The formulation of lip gloss according to claim 1, wherein the Flavors in the formulation is in the range of between 0.01-0.1% by weight.

27. The formulation of lip gloss according to claim 1, wherein the said formulation may further comprise at least one of the following additives: CI 15850, CI 19140, CI 17200, CI 15985, CI 42090, CI 15510 and CI 16035 in specified amount by weight.

28. The formulation of lip gloss according to claim 27, wherein the said specified amount the additives is in the range of between 0.01-01% each by weight.

29. The formulation of lip gloss according to claim 27, wherein the said additive formulation further comprises characteristics of shiny finish, long lasting or transfer resistance.

30. The formulation of lip gloss according to claim 1, wherein the plurality functional ingredients within the formulation further comprises; solvent, emulsifier, emulsifying stabilizer, skin feel regulators, antioxidants, emollients, moisturizer, aromatics and colorant.

31. The formulation of lip gloss according to claim 30, wherein the ingredient with solvent function is about 65% by weight.

32. The formulation of lip gloss according to claim 30, wherein the ingredient with emulsifier about 2% by weight.

33. The formulation of lip gloss according to claim 30, wherein the ingredient with emulsifying stabilizer function is in the range of between 0.56-2.1% by weight.

34. The formulation of lip gloss according to claim 30, wherein the ingredient with skin feel regulator function is in the range of between 0.1-5% by weight.

35. The formulation of lip gloss according to claim 30, wherein the ingredient with antioxidants function is in the range of between 0.1-0.2% by weight.

36. The formulation of lip gloss according to claim 30, wherein the ingredient with emollients function is in the range of between 8-11% by weight.

37. The formulation of lip gloss according to claim 30, wherein the ingredient with moisturizer function is in the range of between 0.05-0.2% by weight.

38. The formulation of lip gloss according to claim 30, wherein the ingredient with aromatics function is about 0.03% by weight.

39. The formulation of lip gloss according to claim 30, wherein the ingredient with colorant function is in the range of between 0.1-0.3% by weight.
